(19) United States
(12) Patent Application Publication
(10) Pub. No.: US 2012/0283828 A1
(43) Pub. Date: Nov. 8, 2012

(54) NON-WOVEN FABRIC FOR MEDICAL USE AND PROCESS FOR THE PREPARATION THEREOF

(75) Inventors: Frank Willems, Moers (DE); Christoph Classen, Monschau (DE); Stefanie Kaempf, Aachen (DE)

(73) Assignee: NonWoTecc Medical GmbH, Koln (DE)

(21) Appl. No.: 13/508,299

(22) PCT Filed: Nov. 5, 2010
(86) PCT No.: PCT/EP1200/066928

§ 371 Date: Jul. 20, 2012

(30) Foreign Application Priority Data
Nov. 5, 2009 (EP) 09175166.9
Jun. 2, 2010 (EP) 10164722.0

(51) Int. Cl. A61F 2/02; B29C 41/08

(52) U.S. Cl. 623/11.11; 264/309

(57) ABSTRACT

Process for preparing a non-woven fabric for medical use, comprising the following steps: —performing a spraying process in which —a spray jet is generated from a polycarbonate urethane plastic solution having a viscosity of from 800 to 1500 Pa×s using a spraying unit from which the polycarbonate urethane plastic solution exits; —said spray jet includes at least one strand of a single microfiber that may have a diameter of from 1 to 15 μm, preferably a diameter of from 2 to 10 μm; —said at least one strand of the single microfiber is sprayed onto a support, wherein —the support is moved relative to the spraying unit, or the spraying unit is moved relative to the support; —the spraying process is repeated several times to form polycarbonate urethane microfiber layers; —whereby polycarbonate urethane microfibers formed from the individual spraying processes overlay each other and adhere to each other at the respective contact points; and —a non-woven fabric having a fibrillar microporous structure is formed; —and optionally detached from the support after the last spraying process.

The invention relates to a non-woven fabric for medical use, a process for preparing the non-woven fabric according to the invention, especially a non-woven fabric consisting of interconnected fine-fibrillar fibers, for example, of polyurethane, and uses of the non-woven fabric.

A process for preparing non-woven fabrics from polyurethanes is described in DE-A-28 06 030. In this process, polyurethanes are dissolved in solvents, such as dimethylformamide, acetone or toluene, and spun into microfibers using an automated spraying device. The microfibers formed are applied to a rotating molded part layer by layer at defined angles, and bonded and fused together layer by layer at the crossing sites. With the process described, non-woven fabrics having a microporous structure can be prepared. Such a medical non-woven fabric could be employed, in particular, in the preparation of vascular prostheses, since the basic mechanical and biological properties of the material roughly meet the requirements for such a prosthesis.

A basic requirement for an implantable product is to ensure an appropriate biocompatibility. In the special case of vascular prostheses, it is additionally necessary that the product is biostable over the period of application. Further, the properties are described as being advantageous if the material used for a vascular prosthesis is elastic, promotes physiological colonization by cells and minimizes the risk of track bleeding, which often occurs during surgery. It is a particular challenge to meet the entirety of these properties of a product, which in part have contrary effects.

Therefore, no product on the basis of the non-woven fabric described above that has proven to display long-term stability in long-term tests in both its mechanical and biological properties and thus meets the requirements for a permanent graft has been commercially available to date. All polyurethanes used to date are subject to chemical alterations in long-term tests, which have the effect that the mechanical properties of the material or the structure deteriorated and resulted in a failure of the prosthesis (dilatation).

For 20 years, it has been true that artificial vascular prostheses are almost exclusively woven or knitted from PET yarns (Dacron) or extruded from PTFE (Teflon). Knitted prostheses tend to dilate, and woven prostheses, although more dimensionally stable, are relatively rigid. Woven and knitted prostheses are often finished (coated) with collagen, albumin or gelatin, or “conditioned” with the patient's own blood before the grafting. Extruded PTFE forms a dense and inert wall. Expanded PTFE (ePTFE), which has been introduced about 15 years ago, can be more or less porous.

Woven prostheses are preferably used for replacing the aorta in the thoracic region. Due to their structure, woven prostheses are little elastic, which can affect the so-called windkessel function. The windkessel function enables the arterial blood to continuously flow in the peripheral vessels. A disorder of the windkessel function would result in an enhanced work performance by the heart, which may lead in a damage to the heart in the long term.

Knitted vascular prostheses are preferably employed in the abdominal and peripheral regions. Due to their tendency to dilatation, they cannot be employed in a region close to the heart, where higher pressures generally prevail.

Since both woven and knitted vascular prostheses have a high porosity, such prostheses must be sealed (“preclotted”) before the grafting. A preferred method is the sealing of the prosthesis with albumin, collagen or gelatin in denatured form. This has the advantage that the prosthesis can be employed immediately. Another method involves the soaking of the prosthesis in the patient's own blood. The blood will penetrate the structure and pores of the prosthesis and coagulate after some time. After the grafting, integration into the connective tissue occurs. An excess growth of endothelial cells in the region of the connecting sites frequently occurs on the side facing towards the blood. This results in a reduction of the free lumen and thus a reduction of the blood flow. For small interior diameters of the prosthesis, this may very quickly lead to an obstruction of the prosthesis. Therefore, woven and knitted prostheses are employed only for vessels having large interior diameters and high flow rates, mainly in the thoracic and abdominal regions.

Vascular prostheses of ePTFE are employed, in particular, as a substitute for small vessels, especially in the coronary region. However, they have little elasticity. Due to the smoothness of the surfaces, integration into the connective tissue does not occur from either the inside or the outside. In the exterior region, encapsulation occurs since the body considers the prosthesis as an inert foreign body. In the interior region, a neointima is formed again and again at the connecting sites, but the cells cannot hold on the smooth surface. Prostheses made of ePTFE tend to track bleedings, which may lead to complications during surgery and extends the operation time.

An object of the invention is to provide a medical product that meets the requirements stated above and avoids the drawbacks described above, and to provide a process for the preparation thereof.

This object is achieved by the process according to the invention and novel non-woven fabrics having a defined non-woven structure obtainable thereby, for medical use.

According to the invention, a process is described for preparing a non-woven fabric for medical use, comprising the following steps:
performing a spraying process in which
   a spray jet is generated from a polycarbonate urethane plastic solution having a viscosity of from 800 to 1500 Pa·s using a spraying unit from which the polycarbonate urethane plastic solution exits;
   said spray jet includes at least one strand of a single microfiber;
   said at least one strand of the single microfiber is sprayed onto a support, wherein
the support is moved relative to the spraying unit, or the spraying unit is moved relative to the support;
the spraying process is repeated several times to form microfiber layers;
whereby microfibers formed from the individual spraying processes overlay each other and adhere to each other at the respective contact points; and
a non-woven fabric having a fibrillar microporous structure is formed;
and optionally detached from the support after the last spraying process.

In connection with the non-woven structure according to the invention, the polycarbonate urethane used in the process according to the invention surprisingly leads to a medical product having long-term stability that meets the mechanical and biological requirements for a long-term implant.

The non-woven fabric obtainable by the process according to the invention can be used, in particular, in medical engineering for vascular prostheses, tissue patches or as a cell culturing matrix.

As the solvent in the preparation process according to the invention for the preparation of the plastic solution, there may be used at least one organic solvent, especially a halogenated solvent. Particularly preferred are solvents such as dimethylacetamide, tetrahydrofuran or chloroform. The plastics employed are dissolved in the above mentioned solvents at concentrations of from 5 to 15%. The solutions prepared are subjected to a repeated temperature treatment in order to ensure an optimum solution of the plastic in the solvent.

Further, a process for the preparation is described that enables to prepare non-woven fabrics having defined biomechanical properties, depending on the intended later use.

Under the process aspect, the object of the invention is achieved by a facility construction in which the individual parameters of the process are exactly adjustable. Since the generation of the non-woven structure is dependent on geometric parameters (e.g., distance between automated spray device and support), atmospheric parameters (e.g., temperature or atmospheric humidity) and kinematic parameters (e.g., coating rate), the adjustment is effected by a Cartesian multiaxial system or an atmospherically encapsulated, temperature-adjustable production chamber.

In the process according to the invention, polycarbonate urethane is employed as the plastic materials. The plastic material is employed, for example, at a concentration of from 5 to 15% by weight in the solution.

To facilitate the application, for example, as a vascular prosthesis, it may be advantageous to apply a visually discernible orientation element to the underlying layers of polycarbonate urethane microfibers in later spray operations or after the last spray operation. This can be achieved, for example, by a differently dyed polycarbonate urethane microfiber.

The porosity of the individual layers formed by the microfiber layers can be adjusted by suitable measures. These include, in particular, the selection of a suitable application distance, an application rate, adjustment of the feed pressure of the solution, the spraying pressure and solvent concentration or the viscosity of the solvent in view of the desired non-woven structure.

The flight time and thus the drying time of the fibers during the flight can be changed by changing the application distance. When the application distance is great, the fibers have a low residual moisture of solvent when hitting the molded part, and when the application distance is small, the fibers have a rather high residual moisture of solvent when hitting the substrate. The higher the residual moisture of the fibers, the more and the stronger fusing points form an the contact sites of the individual fibers. As the flight time is shortened, the velocity with which the fibers hit the molded part increases. During the drying and the accompanying setting processes of the structure, this results in an increase of the compactness of the structure and thus in a reduction of porosity.

The velocity of the application is combined from the velocity along the rotational axis (axial direction) and the circumferential velocity of the molded part (circumferential direction), which is calculated from the rotation rate and the circumference of the molded part. The application density can be increased by increasing the circumferential velocity or reducing the velocity along the rotational axis.

In molded parts whose diameters are larger than the diameter of the impinging spray jet, the orientation of the fibers is affected. The orientation of the fibers on the molded part is generally dependent on the preset spraying angle. This is normally at 45°, with respect to the rotational axis. By mutually adjusting the two velocities, a preferential direction of the laid fibers and thus the material performance of the structure in the longitudinal and transversal directions can be adjusted. For a homogeneous structure, the two velocities must be the same. If a higher tensile strength in an axial direction is demanded as compared to the circumferential direction, the velocity in axial direction must be higher than the velocity in circumferential direction. Conversely, the tensile strength in circumferential direction is higher if the velocity in circumferential direction is higher than the velocity in axial direction. This is interesting for use in vascular surgery, since the non-woven structure can be matched to the orientation of the different layer structures of the vascular wall.

The feed pressure and the spray pressure must be matched to the polymer solution employed and the polymer employed. Depending on the required fiber structure, the material feed rate and thus the material feed pressure and the spray pressure are adjusted. Increasing the material feed pressure and reducing the spray pressure renders the fibers thicker in total, or fiber strands are increasingly formed. Conversely, thin fibers are increasingly formed if the material pressure is reduced and the spray pressure increased.

The feed pressure is typically within a range of from 250 to 1500 mbar, and the spray pressure is from 500 to 3000 mbar, for example.

The support that can be employed in the process according to the invention is essentially planar, conical-frustum shaped, conical or cylindrical. It may be advantageous to provide the support with a surface that is solvent-resistant and enables the detachment of the product from the support after drying, in particular. In the simplest case, the support consists of a material that has these surface properties. The following materials may usually be employed: polyethylene (PE), polyamide (PA) or polytetrafluoroethylene (PTFE).

A non-woven fabric for medical use can be prepared by the process according to the invention.

In one embodiment, it is a tubular non-woven fabric having inner and outer surfaces that can be prepared by a process in which the spraying is effected onto a substantially cylindrical support. In particular, the tubular non-woven fabric according to the invention may have a porosity that essentially allows the liquid components of blood to pass while it essentially retains the cellular components of blood. An advantage thereof is the fact that an autonomous ventilation may occur with simultaneous sealing against non-gaseous components. For example, after the beginning of blood flow through the implanted prosthesis, air contained therein may escape through the pores while the liquid and solid components can penetrate the pores only partially or not at all.

In particular, the tubular non-woven fabric according to the invention may be provided with an inner surface structure of polycarbonate urethane microfiber layers that has a finer structure as compared to the outer surface structure, so that the ratio of the pore size on the lower side to the pore size on the upper side is 1:50, especially 2:10 and preferably 4:8.

According to the invention, the layer of non-woven material has fibers with a diameter of from 0.1 μm to 100 μm, especially from 0.2 μm to 20 μm, and preferably from 0.3 μm to 1 μm.

According to the embodiment of the invention, the layer of non-woven material has a lower side and an opposing upper side. Further, the layer of non-woven material has pores on its upper side that have a size other than that of the pores on the lower side. According to the invention, the pores on the lower side of the layer of non-woven material are smaller than the pores on the upper side of the layer of non-woven material. The ratio of the pore size on the lower side to the pore size on the upper side is 1:50, especially 2:10 and preferably 4:8.

The layer of non-woven material has a thickness of from 10 μm to 3000 μm.

According to an advantageous embodiment of the invention, the layer of non-woven material is stretchable, wherein the thickness of the layer of non-woven material before the stretching is from 100 μm to 3000 μm, preferably from 150 μm to 2800 μm, and especially from 200 μm to 2000 μm.

According to another advantageous embodiment of the invention, the layer of non-woven material is stretchable, wherein the thickness of the layer of non-woven material after the stretching is from 10 82 m to 2500 μm, preferably from 20 μm to 2000 μm, and especially from 80 μm to 1000 μm.

In another embodiment, the microfiber layers forming the inner surface or being close to the inner surface in the tubular non-woven fabric according to the invention have a porosity that is smaller than the porosity of the microfiber layers forming the outer surface or being close to the outer surface.

In still another embodiment of the tubular non-woven fabric according to the invention, it has an inner surface that enables or facilitates colonization by endogenous neointima cells.

The tubular non-woven fabric according to the invention may also be provided with an outer surface that enables or facilitates integration into the connective tissue.

This may be achieved by a selective adjustment of the preparation parameters, whereby layers having different properties can be prepared.

In vitro experiments showed that the non-woven fabrics according to the invention promote the tendency of certain cell types, such as stem or progenitor cells or blood vessel wall cells, to colonize their surfaces. For this purpose, cell culture experiments were performed in direct contact with the outer and inner surfaces, e.g., tubular non-woven fabrics, and evaluated by microscopy.

Upon direct contact, the cells in a suspension were charged onto the surface of the non-woven structure, and a microscopic evaluation was subsequently performed. Glass was used as a negative control, and a polyvinyl chloride surface was used as a positive control. The cell growth on the non-woven fabric had an optimum growth rate that was identical to that of the glass negative control. The PVC positive control showed a complete inhibition of cell growth, which confirms the validity of the test.

This selective 3D structurization, which is essentially defined by the pore size and shape and the connection between the pores, enables, for example, the preferred colonization of the finer inner layer by endothelial cells while colonization by adventitia and muscle cells hardly occurs there. Conversely, neointima cells will hardly colonize on the coarser outer surface, while the adventitia and muscle cells preferentially colonize here.

In a further embodiment of the tubular non-woven fabric according to the invention , it is provided with an orientation line arranged on the outer surface in the longitudinal direction of the tubular non-woven fabric.

The tubular non-woven fabric according to the invention may also be provided with an interior diameter that decreases in the longitudinal direction of the tube, especially with a conicity that is essentially linear throughout the length of the non-woven fabric.

In another embodiment of the invention, the non-woven fabric is in the form of a patch-like non-woven fabric and is obtainable by a process in which the spraying is effected onto a substantially planar support.

The use of the patch-like non-woven fabric according to the invention for, among others, angioplastic or neurosurgical reconstruction, for example, of the dura mater, is claimed according to the invention.

The patch-like non-woven fabric according to the invention may also be used for in-vitro colonization by human cells for the culturing of body tissue.

The tubular non-woven fabric according to the invention may also be employed as a vascular prosthesis, as a suture ring, especially as a cardiac valve suture ring, as a suture ring for cannulas used for heart support systems, with a non-woven-like fine fibrillar structure, and also as a germ barrier for cannulas used in extracorporeal heart support systems.

The non-woven fabric with its structure of fine fibrillar fibers is particularly suitable for colonization by and culturing of endogenous cells. By changing different parameters in the preparation process, non-woven structures having different properties, mechanical and biological, appropriate for the respective intended use, can be prepared.

Thus, the non-woven fabric according to the invention can be used in medical engineering for:

Vascular prostheses, angioplasty patches, neuroplasty patches, blood filters, cell colonization matrix, oxygenator matrix, use in veterinary medicine, coating of coronary stents, suture rings for cardiac valves, coating for VAD cannulas, suture rings for VAD cannulas, cover patch (obturator) in root canal treatment, germ barrier for PEG probe, VAC (vacuum assist closure therapy) patch.

Further fields of application are in the fields of vascular surgery, neurosurgery, cardiosurgery, plastic surgery, regenerative medical engineering (tissue engineering) and for heart support systems.

Another possible application is the use of the non-woven structure for the deposition of medicaments. These may be delivered in the place of use over an extended period of time (drug-eluting implant). In this case, the drugs may be encapsulated, for example, in capsules of lactic acid compounds that dissolve over time. the deposition of silver ions acting as anti-inflammatory agents is also possible. This is also interesting in view of the fact that when inflammations occur at the vascular prosthesis, always the complete prosthesis must be removed.

The use of a non-woven fabric of polyurethane has numerous advantages. The non-woven structure shows elastic behavior similar to that of human tissue. Due to the porous structure, there is more or less intensive growing of cells into the non-woven structure without essentially changing the behavior of the material. When used as a vascular prosthesis, the structure can be designed in such a way that a compact, but structured surface that favors the formation of a neointima of myofibroblasts or endothelial cells is formed inside. The surrounding non-woven structure is designed to enable the growing in of fibroblasts and minute vessels (vasa vasorum), which are important for supplying the neointima.

Surprisingly, it could be found within the scope of this invention that the vascular prostheses prepared according to the processes described above did not show any alteration in the structure and the mechanical characteristics after completion of several months of animal tests.

1-16. (canceled)

17. A process for preparing a non-woven fabric for medical use, comprising:
performing a spraying process comprising
generating a spray jet of a polycarbonate urethane plastic solution having a viscosity of from 800 to 1500 Pa·s using a spraying unit from which the polycarbonate urethane plastic solution exits, wherein
the spray jet includes at least one strand of a single microfiber,
the at least one strand of the single microfiber is sprayed onto a support, and
the support is moved relative to the spraying unit or the spraying unit is moved relative to the support; and
repeating the spraying process to form polycarbonate urethane microfiber layers;
wherein the polycarbonate urethane microfibers formed from the individual spraying processes overlay each other and adhere to each other at points of contact between the microfibers; and
a non-woven fabric having a fibrillar macroporous structure is formed.

18. The process of claim 17 wherein the at least one strand of a single microfiber has a diameter from 1 to 15 μm.

19. The process of claim 17 further comprising detaching the fabric from the support after the last spraying process.

20. The process according to claim 17 wherein said polycarbonate urethane plastic material is in a concentration of from 8% by weight to 12% by weight.

21. The process according to claim 20 wherein the polycarbonate urethane material employed has an average molecular weight (M_{w}) of from 90,000 to 150,000 g/mol.

22. The process according to claim 17 further comprising applying a visually discernible orientation element to underlying layers of polycarbonate urethane microfibers during or after the spraying process.

23. The process according to claim 17 wherein a porosity of each the individual layers formed by the microfiber layers is adjusted.

24. The process according to claim 17 wherein an organic solvent is used as solvent for the polycarbonate urethane plastic solution.

25. The process according to claim 17 wherein the support is essentially planar, conical-frustum shaped, conical or cylindrical.

26. A non-woven fabric for medical use made by the process of claim 17.

27. A tubular non-woven fabric having inner and outer surface structures made by the process of claim 17, wherein said spraying operation is effected onto a substantially cylindrical support.

28. The tubular non-woven fabric according to claim 27, having a porosity that essentially allows the liquid components of blood to pass while essentially retaining cellular components of the blood.

29. The tubular non-woven fabric according to claim 28 comprising an inner surface structure of polycarbonate urethane microfiber layers that has a finer structure as compared to an outer surface structure, so that a ratio of a pore size on the inner side to a pore size on the upper side is 1:50, especially 2:10 and preferably 4:8.

30. The tubular non-woven fabric according to claim 29 having several layers that are different from each other in terms of thickness and porosity and enablement of different cell types to grow in or immigrate.

31. The tubular non-woven fabric according to claim 29 having an inner layer with a thickness of from 10 to 90 layers, and having an outer layer with a thickness of from 10 to 100 layers,

32. The tubular non-woven fabric according to claim 31 wherein the surface of the inner layer enables or facilitates colonization by endogenous intima and/or endothelial cells and wherein the surface of the outer layer enables or facilitates colonization by endogenous adventitia and muscle cells.

33. A patch-like non-woven fabric made by a process according to claim 17 wherein the spraying is effected onto a substantially planar support.

34. A method of angioplastic or neurosurgical reconstruction comprising implantation of the patch-like non-woven fabric of claim 33.

35. A tubular non-woven fabric made by a process of claim 17 formed as device chosen from the group consisting of a vascular prosthesis, a suture ring, a cardiac valve suture ring, a suture ring for a cannula used for a heart support system, a device having a non-woven-like fine fibrillar structure, and a germ barrier for cannulas used in extracorporeal heart support systems.

36. A medical device comprising a plurality of layers, with the layers comprising polycarbonate urethane microfibers, wherein the layers forming a non-woven fabric with a fibrillar microporous structure.
